(19) United States
(12) Patent Application Publication
(10) Pub. No.: US 2011/0263858 A1
(43) Pub. Date: Oct. 27, 2011

(54) PROCESS FOR THE PREPARATION OF CLOPIDOGREL HYDROGEN SULPHATE FORM I

(76) Inventors: Satish Kumar Aryan, Ambala City (IN); Parendu Dhirajlal Rathod, Gurgaon (IN); Ram Chander Aryan, New Delhi (IN); Chandra Has Khanduri, Gurgaon (IN)

(21) Appl. No.: 13/125,114

(22) PCT Filed: Oct. 20, 2009
(86) PCT No.: PCT/IB09/54626

§ 371 Date: May 25, 2011

(30) Foreign Application Priority Data
Oct. 20, 2008 (IN) 2395/DEL/2008

(51) Int. Cl. C07D 495/04

(52) U.S. Cl. 546/114

(57) ABSTRACT

The present invention relates to a process for the preparation of Form 1 of (+)-(S)-∞-(2-Chlorophenyl)-6,7-dihydrothieno[3,2-c]pyridine-5(4H)-acetic acid methyl ester hydrogen sulphate commonly known as clopidogrel bisulphate. The present invention further relates to a process for reducing the residual amount of methyl isobutyl ketone and controlling the amount of mesityl oxide in clopidogrel hydrogen sulphate Form 1 by washing with ethyl acetate. Formula (I).

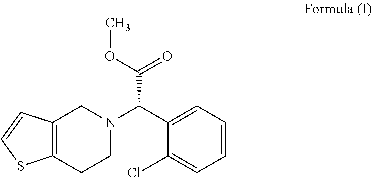

### FIELD OF THE INVENTION

The present invention relates to a process for the preparation of Form 1 of (+)-(S)-α(2-Chlorophenyl)-6,7-dihydrothieno[3,2-c]pyridine-5(4H)-acetic acid methyl ester hydrogen sulphate commonly known as clopidogrel bisulphate. The present invention further relates to a process for reducing the amount of residual methyl isobutyl ketone and controlling the amount of mesityl oxide in Clopidogrel hydrogen sulphate Form 1 by washing with ethyl acetate.

### BACKGROUND OF INVENTION

(+)-(S)-α(2-Chlorophenyl)-6,7-dihydrothieno[3,2-c]pyridine-5(4H)-acetic acid methyl ester of formula (I) known as clopidogrel is an inhibitor of ADP-induced platelet aggregation acting by direct inhibition of adenosine diphosphate (ADP) binding to its receptor and of the subsequent ADP-mediated activation of the glycoprotein GPIIb/IIIa complex.

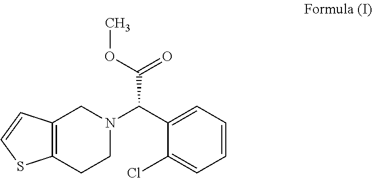

Clopidogrel is administered as its hydrogen sulphate (bisulphate) salt. Clopidogrel hydrogen sulfate has an empirical formula of C₁₆H₁₆CINO₂S.H₂SO₄ and is currently being marketed as PLAVIX® tablets, which contain 97.875 mg of clopidogrel hydrogen sulphate which is the molar equivalent of 75 mg of clopidogrel base.

Clopidogrel hydrogen sulphate is a white to off-white powder. It is practically insoluble in water at neutral pH but freely soluble at pH 1. It also dissolves freely in methanol, dissolves sparingly in methylene chloride, and is poorly in ethyl ether.

U.S. Pat. No. 4,847,265 provides (+)-(S)-∞-(2-Chlorophenyl)-6,7-dihydrothieno[3,2-c]pyridine-5(4H)-acetic acid methyl ester and a pharmaceutically acceptable salt thereof. The hydrochloride, hydrogen sulfate, hydrobromide and taurocholate salts are specifically provided.

PCT Publication No. WO 99/65915 (hereinafter referred as “WO '915”) provides two polymorphs of clopidogrel hydrogen sulphate, referred to as Form 1 and Form 2, though Form 1 is originally disclosed in U.S. Pat. No. 4,847,265. According to WO '915, Form 1 has a XRD pattern with d-spacing (Å) at 9.60, 3.49, 3.83, 3.80, 4.31, 8.13, 4.80, 3.86, 5.80, and 4.95. Both Form 1 and Form 2 are crystallized from acetone under different conditions. WO '915 further provides that clopidogrel hydrogen sulphate Form 2 is thermodynamically more stable than Form 1.

PCT Publication No. WO 03/051362 provides crystalline forms of clopidogrel hydrogen sulphate, referred to as form III, IV, V, VI and amorphous clopidogrel hydrogen sulphate and processes for their preparation. These crystalline forms of clopidogrel hydrogensulfate are solvates of various solvents. Clopidogrel hydrogensulfate Form III is a solvate of 1-butanol, and contains about 7 to about 8% 1-butanol by weight. Form IV is considered a solvate of isopropanol, and contains about 3% to about 9% isopropanol by weight. Form V is a solvate of 2-butanol and contains about 9% to about 10% 2-butanol by weight. Form VI is a solvate of 1-propanol, and contains about 6% propanol by weight.

PCT Publication No. WO 04/020443 provides a process for the preparation of clopidogrel hydrogen sulphate Form 1 which involves dissolving clopidogrel base in isopropanol and/or butyl acetate, cooling the mixture, adding sulphuric acid and inoculating the mixture with Form 1 of clopidogrel hydrogen sulphate and stirring the crystallized mixture at a temperature between −5 and 15° C. to get crystals of clopidogrel hydrogen sulphate Form 1.

PCT Publication No. WO 2004/048385 provides a process for the preparation of clopidogrel hydrogen sulphate Form 1 which involves dissolution of clopidogrel base in a precipitating solvent such as 1,2-dimethoxyethane, 1,2-diethoxyethane, t-butyl methyl ether, bis(2-ethoxyethyl)ether, dioxane, isobutyl methyl ketone, bringing the solution thus obtained to a temperature of 0° C., adding sulphuric acid drop wise, stirring the suspension at 0 to 5° C. for 12 hours and isolating Form 1 of clopidogrel hydrogen sulphate from the reaction mixture.

PCT Publication No. WO 2005/104663 provides a process for the preparation of clopidogrel hydrogen sulphate Form 1 which involves dissolving clopidogrel base in a solvent such as methyl propyl ketone, methyl isopropyl ketone, diethyl ketone or their mixtures, mixture of ethyl acetate and methyl propyl ketone, mixture of ethyl acetate and methyl isopropyl ketone, or mixture of ethyl acetate and diethyl ketone, cooling the solution to a temperature of about −10 to 20° C., adding concentrated sulphuric acid to the cooled solution; maintaining the salt mixture at a temperature in the range of about 10 to 30° C. to effect precipitation and filtering Form 1 of clopidogrel hydrogen sulphate from the mixture.

PCT Publication No. WO 2007/125544 provides a process for the preparation of clopidogrel hydrogen sulphate Form 1 which involves dissolving clopidogrel base in suitable organic solvent such as methyl isobutyl ketone, n-hexane, n-heptane, adding halogenated solvent such as methylene chloride, chloroform, carbon tetrachloride, ethylene dichloride and seeding Form 1 of clopidogrel hydrogen sulfate, cooling the reaction mixture to −10 to 0° C., adding solution of sulfuric acid in methyl isobutyl ketone, maintaining the temperature below 0° C., stirring the reaction mixture for sufficient time to convert to Form 1 of clopidogrel hydrogen sulfate, isolating clopidogrel hydrogen sulfate Form 1.

Chinese Patent Application No. CN 1903859 provides a method of preparation of clopidogrel hydrogen sulphate Form 1 which dissolving clopidogrel free base in a ketone solvent selected from 5 carbon ketone or 6 carbon ketone, cooling the mixture to −15 to 25° C., adding sulphuric acid drop wise, raising the temperature to 20 to 50° C. and maintaining this temperature stirring for ½ to 3 hours, isolating, and drying the wet product to get Form 1 of clopidogrel hydrogen sulphate.

PCT Publication No. WO 2008/019053 provides a process for the preparation of clopidogrel hydrogen sulphate Form 1 which involves, combing a solution of clopidogrel base in methyl isobutyl ketone and a suspension of clopidogrel hydrogen sulphate in methyl isobutyl ketone at ˜—10° C. to obtain a suspension, adding sulphuric acid and stirring the suspension for 17 hours at −10° C., isolating, and drying the wet product to get Form 1 of clopidogrel hydrogen sulphate.

Several processes have been reported for the preparation of clopidogrel or its salt and various polymorphic forms for example in U.S. Pat. No. 5,204,469; Chinese Patent Application Nos. CN 1850827 A; CN 1840533 A; CN 1775782 A; PCT Publication Nos. WO 9851681; WO 9851682; WO 9839322; WO 9918110; WO 03000636; WO 03093276; WO 9851689; WO 0027840; WO 02059128; WO 200218357; WO 2003035652; WO 2003004502; WO 2004013147; WO 2004074215; WO 2004072085; WO 2004072084; WO 2004108665; WO 2004094374; WO 2004081015; WO 2004081016; WO 2004052966; WO 2004026879; WO 2005077958; WO 2005063708; WO 2005016931; WO 2006087729; WO 2005012300; WO 2005003139; WO 2005003138; WO 2005100364; WO 2005113559; WO 2005087779; WO 2006086921; WO 2006137628; WO 2006042481; WO 2006091847; WO 2006087226; WO 2007028337; WO 2006094468; WO 2007032023; WO 2007017886; WO 2007073095; WO 2007074995; WO 2007094006; WO 2007144729; WO 2007144895; WO 2008004249; WO 20080034912.

It is apparent from the prior art that same solvent can give different polymorphs under different experimental conditions. Most of the prior art methods for the preparation of clopidogrel hydrogen sulphate Form 1 from different solvents require very specific temperature range and specific conditions to get reproducible results. Since Form 1 is kinetically controlled form and Form 2 is thermodynamically controlled form, minor variation in the conditions might result in Form 2 or a mixture of Form 1 and Form 2 instead of Form 1.

The present inventors have found that higher residual quantities of methyl isobutyl ketone present in clopidogrel hydrogen sulphate destabilize polymorphic Form 1. Further the use of methyl isobutyl ketone in the process for the preparation of clopidogrel hydrogen sulphate gives rise to the formation of mesityl oxide impurity, which is known to be genotoxic. The present inventors have also found that although washing with acetone though reduced the amount of methyl isobutyl ketone present in clopidogrel hydrogen sulphate Form 1, it also increases the amount of mesityl oxide.

### SUMMARY OF INVENTION

The present inventors have found a process for reducing the amount of methyl isobutyl ketone and controlling the amount of mesityl oxide in Clopidogrel hydrogen sulphate Form 1 which comprises washing clopiodgrel hydrogen sulphate Form 1 isolated from a mixture of methyl isobutyl ketone and clopiodgrel hydrogen sulphate Form 1 with ethyl acetate.

One aspect of the present invention provides a process for preparing clopiodgrel hydrogen sulphate Form 1 comprising:
a) isolating of clopidogrel hydrogen sulphate Form 1 from a mixture of methyl isobutyl ketone and clopidogrel hydrogen sulphate Form 1,
b) washing clopidogrel hydrogen sulphate Form 1 with ethyl acetate, and
c) drying clopidogrel hydrogen sulphate Form 1.
   a)

Another aspect the present invention provides clopidogrel hydrogen sulphate having methyl isobutyl ketone content not more than 900 microgram/gram.

Yet another aspect the present invention provides a pharmaceutical composition that includes a therapeutically effective amount of clopidogrel hydrogen sulphate having less than 900 microgram/gram residual methyl isobutyl ketone; and one or more pharmaceutically acceptable carriers, excipients or diluents.

### DETAILED DESCRIPTION OF THE INVENTION

As used herein “Form 1” refers to Form 1 of clopidogrel hydrogen sulphate disclosed in WO '915, which has a XRD pattern with d-spacing (A) at 9.60, 3.49, 3.83, 3.80, 4.31, 8.13, 4.80, 3.86, 5.80, and 4.95.

In one aspect of the present invention provides a process for preparing clopiodgrel hydrogen sulphate Form 1 comprising:
a) isolating of clopidogrel hydrogen sulphate Form 1 from a mixture of methyl isobutyl ketone and clopidogrel hydrogen sulphate Form 1,
b) washing clopidogrel hydrogen sulphate Form 1 with ethyl acetate and
c) drying clopidogrel hydrogen sulphate Form 1.

The clopidogrel hydrogen sulphate employed in the present process is obtained by dissolving clopidogrel base in methyl isobutyl ketone at 25-30° C., cooling the solution −5 to −10° C., seeding with clopidogrel bisulfate Form 1, adding sulphuric acid at −5 to −10° C., stirring the suspension at −5 to −10° C. for 4 to 6 hours, isolating clopidogrel hydrogen sulphate Form 1.

The clopidogrel base may be prepared by the processes known in the art, for example, from clopidogrel camphorsulfonate salt by neutralizing with an aqueous solution of a weak base, such as sodium bicarbonate or potassium carbonate in the presence of an organic, water-immiscible solvent such as dichloromethane, at the temperature of about 5° C. to about 30° C. The organic layer is separated and concentrated under vacuum to afford clopidogrel free base.

The process can be carried out by dissolving clopidogrel base in methyl isobutyl ketone. The dissolution is carried out at a temperature of about 25° C. to about 30° C. to obtain a solution.

The solution is cooled to a temperature of about −15° C. to about 0° C. and then seeded with clopidogrel hydrogen sulphate Form 1 while maintaining the temperature at about −15° C. to about 0° C., preferably at −10° C. to about −5° C. Seeding with clopidogrel hydrogen sulphate Form 1 provides well-shaped pure crystals of clopidogrel hydrogen sulphate Form 1. The seed of clopidogrel hydrogen sulphate Form 1 may be prepared by the processes known in the art, such as those listed in the Background section of this application.

The reaction mixture is then combined with sulphuric acid at a temperature of about −15° C. to about 0° C. Preferably, the sulphuric acid is concentrated sulphuric acid and is added to the solution gradually. Preferably, the sulphuric acid is added drop-wise to the solution.

After adding the sulphuric acid to the reaction mixture, a suspension of clopidogrel hydrogen sulphate salt is obtained. Preferably, the suspension is stirred for about 2 to about 20 hours at a temperature of about −15° C. to about 0° C.

The salt from the suspension can then be isolated, such as by filtration through filter cloth at a temperature of about −15° C. to about 0° C. under reduced pressure and washed with methyl isobutyl ketone at a temperature of about 0° C. The wet product is then subjected to drying, initially at a temperature of about 25° C. to about 30° C. and then in air oven at a temperature of about 35° C. to about 45° C. The obtained clopidogrel hydrogen sulphate is found to contain methyl isobutyl ketone more than 1250 microgram/gram.

To the dry clopidogrel hydrogen sulphate is added ethyl acetate at about 20° C. to about 25° C. and stirred for about ½ to 1 hour. The solid is recovered by filtration under nitrogen and reduced pressure and then washed with ethyl acetate. The wet product is then subjected to drying at a temperature of about 45° C. to about 55° C. under vacuum.

Another aspect of the present invention provides clopidogrel hydrogen sulphate Form 1 having methyl isobutyl ketone content not more than 900 microgram/gram. More particularly, clopidogrel hydrogen sulphate Form 1 has methyl isobutyl ketone content not more than 835 microgram/gram.

Yet another aspect of the present invention provides a pharmaceutical composition that includes a therapeutically effective amount of clopiodgrel hydrogen sulphate Form 1 having less than 900 microgram/gram methyl isobutyl ketone; and one or more pharmaceutically acceptable carriers, excipients or diluents.

The residual solvent data (microgram/gram) of clopidogrel hydrogen sulphate Form 1, prepared by the process of the present invention is provided Table 1:

TABLE 1

| | | after | after |
|---|---|---|---|
| | after | isolation | isolation |
| | isolation | from MIBK, | from MIBK, |
| | from | washing with | washing with |
| Solvent | MIBK | acetone | ethyl acetate |
| Methanol | 97 | 96 | 102 |
| Acetone | 15 | 180 | 12 |
| Dichloromethane | 6 | ND | 5 |
| Methyl isobutyl | 1289 | 756 | 832 |
| ketone (MIBK) | | | |
| Mesityl oxide | 3 | 26 | 4 |

*ND—Not Detected

While the present invention has been described in terms of its specific embodiments, certain modifications and equivalents will be apparent to those skilled in the art and are intended to be included within the scope of the present invention.

### EXAMPLES

### Example 1

### Preparation of Clopidogrel Hydrogen Sulphate Form 1

Dichloromethane (4 L) and clopidogrel camphorsulfonate salt (1 Kg) were charged at 25-30° C. The mixture was stirred to dissolve at 25-30° C. and then cooled to 10-15° C. Aqueous sodium bicarbonate solution (10&, 4 L) was added slowly gradually, maintaining the temperature at 10-15° C. The mixture was then stirred for 30 minutes at 10-15° C. The layers were separated at 10-15° C. The organic layer was dried over anhydrous sodium sulfate (1 Kg) at 25-30° C. for 10 minutes and filtered at 25-30° C. The solvent was recovered from filtrate completely under vacuum at 25-30° C.

Methyl isobutyl ketone (15 L) was charged to the residue at 25-30° C. and stirred at 25-30° C. till a solution is obtained. The solution was cooled to −5 to −10° C. and seeds of clopidogrel hydrogen sulphate Form 1 (50 g) were charged. Concentrated Sulphuric acid (100 ml) was added drop wise at −5 to −10° C. and stirred at −5 to −10° C. for 5 hours. The solid was filtered through filter cloth at −5 to −10° C. under nitrogen and vacuum. The solid was washed with methyl isobutyl ketone (4 L) at 0° C. and dried at 35-39° C.

Methyl isobutyl ketone content: 1289 microgram/gram.

The dry solid obtained above and ethyl acetate (3.5 L) were charged into a clean and dry round bottom flask at 20-25° C., stirred for 45 minutes, filtered under nitrogen, washed with ethyl acetate (800 ml), and dried under vacuum at 50° C. to obtain the title compound.

Methyl isobutyl ketone content: 832 microgram/gram
Yield: 700 g

1. A process for reducing the amount of methyl isobutyl ketone and controlling the amount of mesityl oxide in clopidogrel hydrogen sulphate Form 1 which comprises,
a) isolating of clopidogrel hydrogen sulphate Form 1 from a mixture of methyl isobutyl ketone and clopidogrel hydrogen sulphate Form 1,
b) washing clopidogrel hydrogen sulphate Form 1 with ethyl acetate.

2. The process according to claim 1 wherein the washing comprises stirring clopidogrel hydrogen sulphate Form 1 with ethyl acetate.

3. The process according to claim 1 wherein the process further comprises drying clopidogrel hydrogen sulphate Form 1, obtained at step (a).

4. A process for the preparation of clopidogrel hydrogen sulphate Form 1 having methyl isobutyl ketone content not more than 900 microgram/gram which comprises,
a) isolating of clopidogrel hydrogen sulphate Form 1 from a mixture of methyl isobutyl ketone and clopidogrel hydrogen sulphate Form 1,
b) washing clopidogrel hydrogen sulphate Form 1 with ethyl acetate.

5. Clopidogrel hydrogen sulphate Form 1 having residual methyl isobutyl ketone content not more than 900 microgram/gram.
